(19) United States
(12) Patent Application Publication
(10) Pub. No.: US 2009/0048526 A1
(43) Pub. Date: Feb. 19, 2009

(54) APPARATUS FOR MONITORING A PERSON'S HEART RATE AND/OR HEART RATE VARIATION; WRIST-WATCH COMPRISING THE SAME

(75) Inventors: Ronaldus Maria Aarts, Eindhoven (NL); Martin Ouwerkerk, Eindhoven (NL)

Correspondence Address: PHILIPS INTELLECTUAL PROPERTY & STANDARDS, 595 MINER ROAD, CLEVELAND, OH, 44143, US

(73) Assignee: KONINKLIJKE PHILIPS ELECTRONICS N. V., Eindhoven (NL)

(21) Appl. No.: 12/097,548

(22) PCT Filed: Nov. 14, 2006
(86) PCT No.: PCT/IB2006/054235

§ 371 Date: Jun. 16, 2008

(30) Foreign Application Priority Data
Dec. 19, 2005 (EP) 05112403.0

(51) Int. Cl. A61B 5/0402

(52) U.S. Cl. 600/508

(57) ABSTRACT

Monitoring apparatus (4) for monitoring a user's heart, the apparatus comprising several sensors (14, 16) for measuring changes in an electrical parameter of a user's arm (20), from which changes in an electrocardiogram, heart rate and/or heart rate variation of the user's heart are determinable. The apparatus further comprises a data processor (28) for determining the electrocardiogram, the heart rate and/or heart rate variation from the changes in the electrical parameter; and an output device (32) for making knowable to the user the electrocardiogram, heart rate and/or heart rate variation. A basic idea of the present invention is, to use only a single wristband, particularly a wrist watch, having all the means to monitor the user's heart, without using for example a chest band. Herein, the single wristband is at least provided with the at least one sensor and particularly also comprises the data processor, and more particularly also comprises the output device.

### FIELD OF THE INVENTION

The invention relates to a monitoring apparatus for monitoring a user's heart, the apparatus comprising: at least one sensor for measuring changes in at least one electrical parameter of one of the user's limbs, particularly an arm, from which changes an electrocardiogram, heart rate and/or heart rate variation of the user's heart are determinable; a data processor for determining the electrocardiogram, the heart rate and/or heart rate variation from the changes in the at least one electrical parameter; and an output device for auditively and/or visually making knowable to the user the electrocardiogram, heart rate and/or heart rate variation.

The invention also relates to a wristwatch comprising such a monitoring apparatus.

Such a monitoring apparatus is widely used by athletes in order to be able monitor heart rate and/or variations in heart rate during performance of endurances sports. Furthermore, such systems are used by heart patients whose heart rate is to be monitored in connection with a possible heart failure.

### BACKGROUND OF THE INVENTION

A system for monitoring a person's heart rate and/or heart rate variation is known. The known system comprises two wristbands. When in operation, each of the wrists of the person whose heart rate is to be determined, wears one of the wristbands, each wristband comprising several sensors, which measure certain electrical parameters from which, an electrocardiogram (ECG) is determined. The system then determines the heart rate and/or changes therein. The wristband has a display on which the heart rate and/or the changes in the heart rate are displayed. Such a system is, for instance, known from ‘High resolution ambulatory electrocardiographic monitoring using wrist-mounted electric potential sensors’ by C. J. Harland, T. D. Clark and R. J. Prance in Measurement Science and Technology, published on May 23, 2003.

Another system is known, which employs electrodes sticking on the surface of a person's body on different locations and a wristband having a display for displaying the heart rate and/or changes in the heart rate.

A disadvantage of the known systems is the fact that the systems are obtrusive for a person using the systems.

A further disadvantage of the system is the fact that data have to be transferred from specific locations on the person's body to the wristband or one of the wristbands in order for the wristwatch to be able to display the heart rate and/or changes therein. If data transfer from the chest belt to the wristwatch is wireless, the data transfer may be disrupted by signals originating from other people using a similar system. If the data is transferred through a wiring, the wiring causes further unnecessary obtrusiveness to the person using the system.

### SUMMARY OF THE INVENTION

An object of the present invention is to address the disadvantages mentioned above.

To achieve the object of the invention, an apparatus is provided wherein the apparatus is configured to position the at least one sensor at or near a surface of the user's limb, particularly to measure the changes in the at least one electrical parameter of the user's limb via the surface of the user's limb.

Using the apparatus according to the invention, all of the sensors can be positioned at a position on or near the limb i.e. at a short distance from each other. This allows for a compact design of the apparatus in contrast with the known system in which an additional chest belt needs to be provided.

Also, because the need for the additional chest belt can be avoided, a design of the apparatus can be provided which design is much more pleasant to wear than the known systems.

Furthermore, owing to the possibility of compact design, interference between the sensors and the data processor are easily avoided.

A preferred embodiment of the apparatus-according claim 2 is provided. At least two sensors are required for performing electrocardiogram (ECG) measurements.

An important advantage of an apparatus according to claim 4 is the fact that electric potential sensors (EP sensors) do not require galvanic contact for performing measurements. For example, two EP sensors can be used, to measure a change in the electric field.

Also, an embodiment of the apparatus according to the invention may be provided, wherein at least one of the sensors comprises an electrode, such as an Ag/AgCl electrode. Because Ag/AgCl electrodes are widely used, using such electrodes can facilitate implementation of the present invention. Furthermore, Ag/AgCl electrodes can provide signals having a good signal/noise ratio.

In another embodiment of the apparatus according to claim 8, more specifically according to claim 9 is provided. Such an embodiment allows for easy construction.

In order to provide the capacitor with a periodic signal, an apparatus according to claim 10 is provided. The periodic signal is preferably a signal substantially consisting of two values. An important advantage thereof can be that no AD converter is required for adapting the output signal of the capacitor for the data processor.

Also, in an embodiment of the apparatus according to claim 14 is provided. This will lead to an improved accuracy of the measurements performed.

In a special embodiment of the present invention the at least one sensor comprises an array of sensors, which also can significantly improve the accuracy of the measurements.

Also, an embodiment of the apparatus according to claim 3 may be provided, which can significantly improve the accuracy of the measurements.

Preferably, the apparatus is configured to position the at least one sensor at the wrist of the user to measure the electrical parameter substantially at the wrist, thus allowing for an even more compact design.

A basic idea of the present invention is to use only a single wristband, particularly a wristwatch, having all the means to monitor the user's heart, without using for example a chest band. Herein, preferably, the single wristband is at least provided with the at least one sensor and particularly also comprises the data processor, and more particularly also comprises the output device.

These and other aspects of the invention will be apparent from and elucidated with reference to the embodiments described hereinafter with reference to the accompanying drawings, in which like reference signs refer to similar parts.

### BRIEF DESCRIPTION OF THE DRAWINGS

In the drawings,

FIG. 1 is a perspective view of a first embodiment of the apparatus according to the present invention;

FIG. 2 is a top view of the apparatus shown in FIG. 1;

FIG. 3 is a top view of a first modification of the embodiment shown in FIG. 1;

FIG. 4 is a top view of a second modification of the embodiment shown in FIG. 1;

FIG. 5 is a first alternate circuitry suitable for implementation in the embodiment shown in FIG. 1;

FIG. 6 is a second alternate circuitry suitable for implementation in the embodiment shown in FIG. 1;

FIG. 7 is a top view of a second embodiment of the apparatus according to the present invention;

FIG. 8 is a top view of a third embodiment of the apparatus according to the present invention; and

FIG. 9 is a schematic of a data processor, as comprised in the apparatus of FIG. 7.

### DESCRIPTION OF PREFERRED EMBODIMENTS

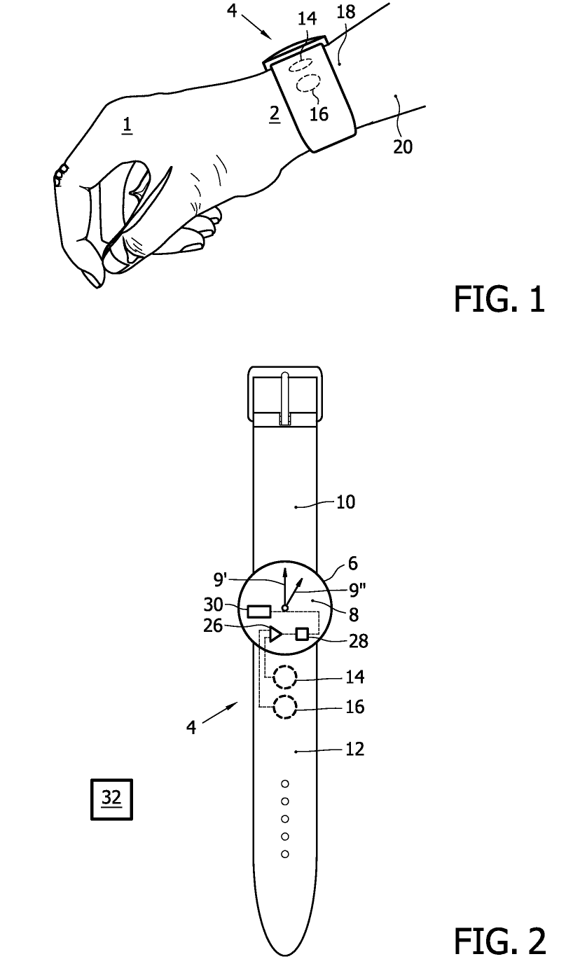

FIG. 1 shows a hand 1 and a wrist 2 of a person using a first embodiment of the monitoring apparatus 4. Referring to FIG. 2, the apparatus 4 comprises a housing 6 having a time display 8 with two pointers 9′ and 9″, and a wrist band having a first strap part 10 and a second strap part 12. For example, the apparatus is a wrist watch-like apparatus, and is configured to be carried by the wrist of the user.

In the present embodiment, the second strap part 12 is provided with two electric potential sensors 14, 16 for determining electric potentials at two different locations on a surface 18 of the wrist 2 of a person's arm 20 (see FIG. 1). As is schematically indicated in FIG. 2 using dotted lines 22 and 24, the sensors 14 and 16 are, in this embodiment, connected with a comparator 26 for determining a difference value from the sensors 14 and 16. The apparatus 4 also comprises a data processor 28 for determining the electrocardiogram, heart rate and/or heart rate variation from the changes in the difference value of the two electric potentials on the surface 18 of the wrist 2. The apparatus 4 further comprises an output device formed by a heart rate display 30 for visually making knowable to the person the electrocardiogram, heart rate and/or heart rate variation. The apparatus is configured to position the at least one sensor at or near a surface of the user's limb, particularly to measure the changes in at least one electrical parameter of the user's limb via the surface of the user's limb, as can be seen in the FIGS. 1 and 2.

An athlete using the apparatus 4 which, in this embodiment, is incorporated in a wrist watch, can, for instance while running, easily check his electrocardiogram, heart rate and/or variations therein by looking at his watch. If the heart rate becomes too high, the athlete can lower his effort in order to maintain a predetermined training scheme.

Alternatively, the apparatus 4 may be used while playing computer games. The electrocardiogram, heart rate and/or heart rate variations may be used as a parameter in computer games. The abilities of a computer game character to be controlled by the user of the apparatus may, for instance, be influenced by the heart rate of the user.

Another application is use of the apparatus, wherein the heart rate and/or heart rate variations are used to determine the occurrence of a heart failure and/or the possibility of occurrence of a heart failure. Upon the determination of a heart failure, the apparatus can be arranged to generate an alarm signal to an external receiver 31.

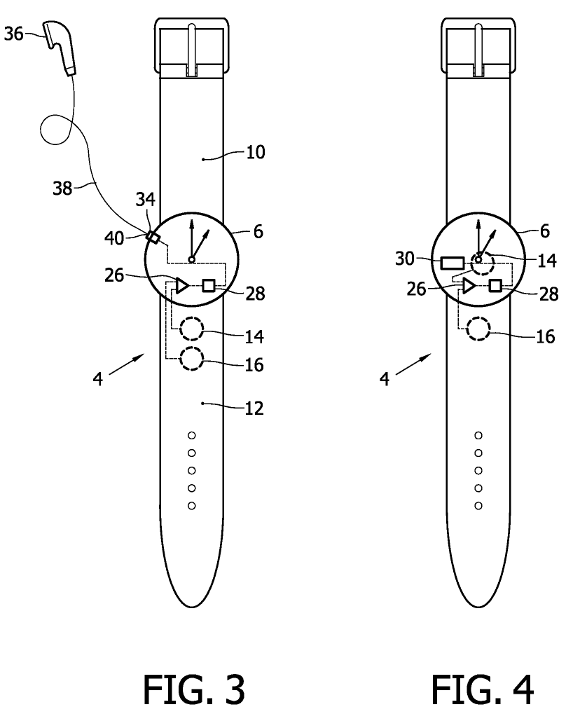

A first modification of the first embodiment is shown in FIG. 3 in which an output device 32 is provided for auditively making knowable to the person the heart rate and/or heart rate variation. Instead of the heart rate display 30, the apparatus 4 comprises an output device formed by a socket 34 and an earpiece 36 having a wiring 38 and a plug 40, which is arranged to be inserted in the socket 34. However, wireless communication between the earpiece 36 and the plug 40 is also possible. In this modification of the first embodiment, the data processor 28 is arranged to periodically notify the person of his/her heart rate and/or heart rate variations by using sound, for instance a human voice.

FIG. 4 shows a second modification of the first embodiment. The second modification differs from the apparatus shown in FIGS. 1 and 2 in that in the second modification, at least one of the electric potential sensors 14 is comprised inside or on the housing 6 of the apparatus 4, which allows for an easy contact between the comparator 26 and the at least one electric sensor 14, because any necessary connection between the electric sensor 14 and the comparator 26 may be entirely inside the housing 6.

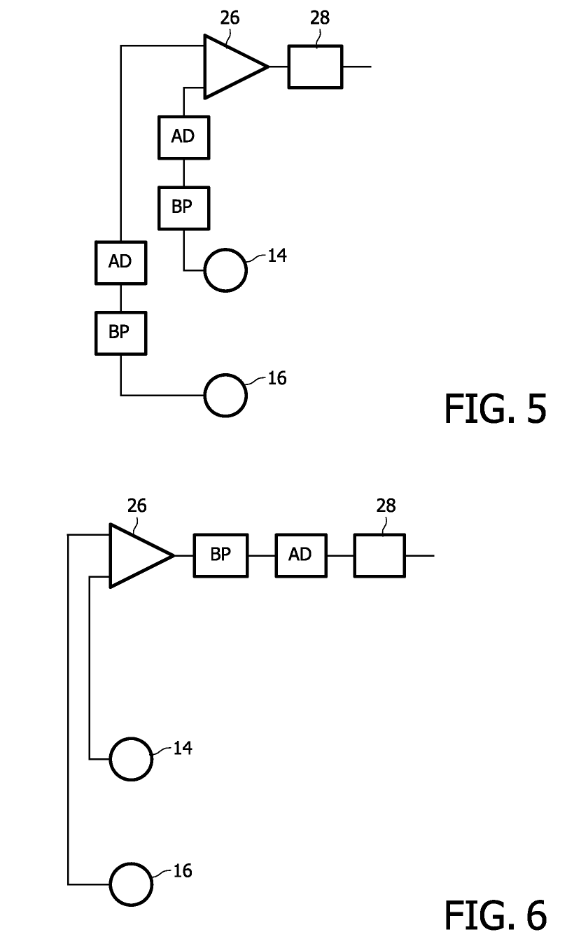

In FIGS. 5 and 6, it is shown that AD converters AD can be provided for converting analogue signals to digital signals. In FIG. 5 each of the sensors 14, 16 is provided with an AD converter directly connected with the respective sensors 14, 16. In FIG. 6, the AD converter AD is positioned, so that the difference value from the sensors 14 and 16 is converted to a digital signal. Preferably, the AD converter comprises a band pass filter BP for suppressing undesired noise.

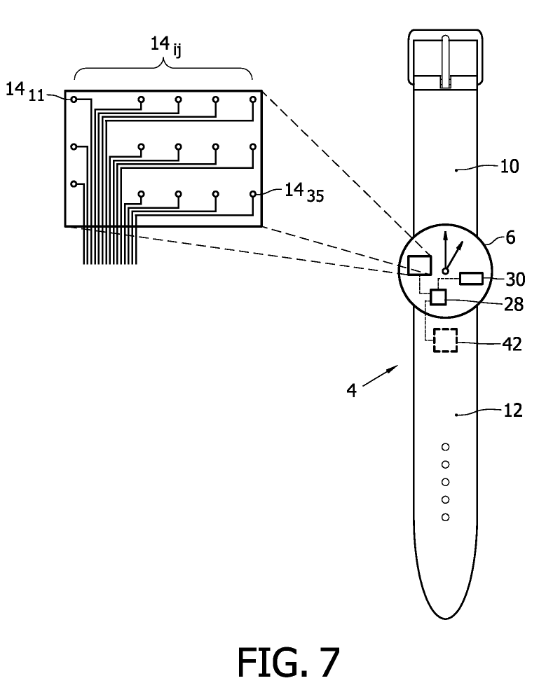

FIG. 7 schematically shows a second, more advanced embodiment of the apparatus. In FIG. 7 the pointers of the time display 8 have been omitted for clarity's sake. In FIG. 6 an array of sensors 14ᵢⱼ is provided. These sensors 14ᵢⱼ are preferably formed by electric potential sensors. However, other sensors may be used as sensors 14ᵢⱼ, such as Ag/AgCl electrodes. In this specific embodiment, the entire array of sensors 14ᵢⱼ is located on the housing 6, in use abutting the surface 18 of the user's arm.

Furthermore, the second embodiment comprises an accelerometer 42, which is provided for determining acceleration of the arm 20. The accelerometer 42 determines an acceleration value of the arm. Also, the data processor 28 is arranged to determine an estimation of a disturbance value in measurements in the at least one electrical parameter caused by the acceleration of the arm in order to be able to compensate for this disturbance. Furthermore, the accelerometer 42 may be arranged to recognize periodic patterns in the acceleration of the arm in order to even further increase the ability to compensation for the disturbance.

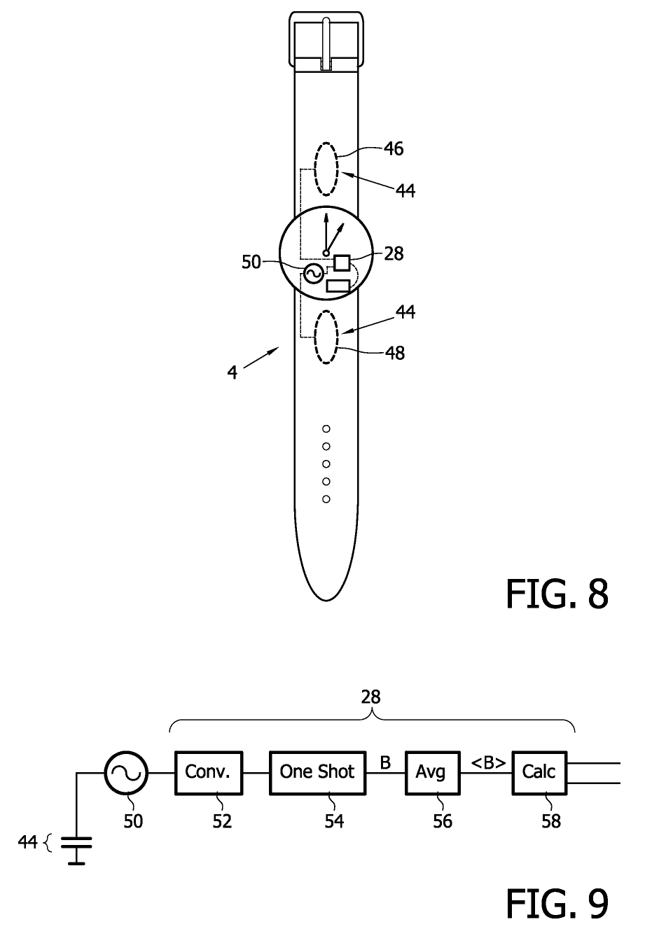

FIG. 8 shows a third embodiment of the apparatus 4. In this embodiment, the apparatus 4 comprises a capacitor 44 having two capacitor electrodes 46 and 48, each of these electrodes being formed by a plate. When in use, the capacitor electrodes 46 and 48 are located at positions on substantially opposite sides of the arm, the opposite sides being faced away from each other at respective positions on or near the arm. The electrodes 46 and 48 are electrically coupled to an oscillator 50, which, in operation, sends an periodic, electrical signal through the capacitor 44 at a certain frequency. In this specific embodiment (see FIG. 9), the data processor 28 of the apparatus 4 further comprises converter 52 for converting the periodic signal to a binary signal A, a one shot 54 for generating a pulse train B as an exit signal in response to a change in signal value of the binary signal and an averaging unit 56 for generating a moving average <B> of the pulse train B generated by the one shot 54. In order to avoid the necessity of the converter 52 an oscillator can be provided arranged to generate a binary signal, such as a block signal. As a single pulse generated by the one shot 52 has a fixed time length and the pulse is generated only in response to a change in the signal value of the exit signal, the moving average value generated by the averaging unit 56 is indicative of the frequency of the binary signal. Based on this moving average signal, the calculating unit 58 can calculate the heart rate and/or heart rate variations.

If a capacity of the capacitor 44 has a constant value, the frequency of the binary signal equals a default frequency of the oscillator. However, when the apparatus 4 is in operation, the capacity of the capacitor 44 varies due to blood pulses occurring in the wrist artery. These blood pulses cause a change in an electrical parameter, such as the overall permittivity of a part of the arm located between the electrodes 46 and 48. As a consequence of this change, the frequency of the binary signal varies and so does the exit value of the averaging unit 56. From this exit value the heart rate and heart rate variation can easily be determined and sent to the display 30.

It will be clear to a person skilled in the art that the invention is not limited to the embodiments shown above. For instance, it is conceivable that the first or the third embodiment is provided with the accelerometer described in the disclosure of the second embodiment. Also, various data processors in addition to the data processors disclosed herein may be suitable for application without departing from the framework of the present invention as determined by the accompanying claims.

For example, the wristband can be configured in various ways, and can include for example one or more straps, and/or be arranged in a different way.

It is once again noted that, by using the apparatus according to the invention, all of the sensors can be positioned at a position on or near the limb i.e. at a short distance from each other. This allows for a compact design of the apparatus in contrast with the known system in which an additional chest belt needs to be provided. Particularly, the apparatus extends substantially around the wrist of the user during use, which allows for an even more compact design.

Also, because the need for the additional chest belt can be avoided, a design of the apparatus can be provided which design is much more pleasant to wear than the known systems.

Furthermore, the at least one sensor, data processor, and/or output device can be coupled to the wristband in various ways, For example, the at least one sensor, data processor, and/or output device can be coupled to the wristband, can be integrally connected to the wristband, can be detachably connectable thereto, can be comprised in a housing of the wristband, and/or in a different manner. Such embodiments are deemed to be included in the scope of the present invention as defined by the accompanying claims.

It is noted that the word “comprising” does not exclude the presence other elements in an part of the apparatus or additional steps in a method. It is also noted that the word “a”/“an” does not exclude plurality. Furthermore, the reference signs in the claims are not to be construed as to limit the scope of the present invention.

1. Monitoring apparatus for monitoring a user's heart, the apparatus comprising: at least one sensor for measuring changes in at least one electrical parameter of one of the user's limbs, particularly an arm, from which changes an electrocardiogram, heart rate and/or heart rate variation of the user's heart are determinable; a data processor for determining the electrocardiogram, the heart rate and/or heart rate variation from the changes in the at least one electrical parameter; and an output device for auditively and/or visually making knowable to the user the electrocardiogram, heart rate and/or heart rate variation, wherein the apparatus is configured to position the at least one sensor at or near a surface of the user's limb, particularly to measure the changes in the at least one electrical parameter of the user's limb via the surface of the user's limb, wherein at least one of the sensors comprises a capacitor having two capacitor electrodes placeable at positions on substantially opposite sides of the limb during use, the at least one electrical parameter comprising a permittivity of the limb, wherein the capacitor is arranged such that in use, a capacitance of the capacitor is sensitive to pressure pulses in at least one blood vessel of the limb.

2. Apparatus according to claim 1, comprising at least two sensors for determining the at least one electrical parameter of the limb.

3. Apparatus according to claim 1, wherein the apparatus comprises a housing, the housing comprising the data processor, the at least one sensor, wherein the housing preferably also comprise the output device.

4. Apparatus according to claim 2, wherein at least one of the sensors is an electric potential sensor.

5. Apparatus according to claim 4, comprising at least two sensors, wherein the apparatus further comprises a comparator for determining a difference value between the values measured by each of the at least two sensors.

6. Apparatus according claim 1, wherein at least one of the sensors comprises an electrode, such as an Ag/AgCl electrode.

7. Apparatus according to claim 1, wherein the apparatus comprises a single wristband, such that the apparatus can be carried by a wrist of the user, wherein the apparatus preferably comprises one wrist watch.

8. (canceled)

9. Apparatus according to claim 1, wherein at least one of the electrodes is formed by a capacitor plate, wherein preferably each of the electrodes is formed by a capacitor plate.

10. Apparatus according to claim 9, wherein the apparatus comprises an oscillator for providing the capacitor with a periodic signal.

11. Apparatus according claim 10, wherein the periodic signal is a signal substantially consisting of two values.

12. Apparatus according to claim 1, wherein the apparatus comprises an accelerometer for determining acceleration of the limb.

13. Apparatus according to claim 12, wherein the data processor is arranged to determine an estimation of a disturbance value in measurements in the at least one electrical parameter which caused by the acceleration of the limb.

14. Apparatus according to claim 13, wherein the data processor is arranged to compensate for the disturbance in the measurements in the at least one electrical parameter.

15. Apparatus according to claim 1, wherein the at least one sensor comprises an array of sensors.

16. Wrist band, for example a wristwatch, comprising a monitoring apparatus according claim 1.

17. Wrist band according to claim 16, wherein the wrist band comprises a time display for displaying time to a person carrying the wrist band on his/her wrist, wherein preferably the output device is comprised in the time display.

18. Use of the apparatus according to claim 1 or the wristband according claim 16, wherein the heart rate and/or heart rate variations are used as a parameter in computer games.

19. Use of the apparatus according to claim 1 or the wrist band according claim 16, wherein the electrocardiogram, heart rate and/or heart rate variations are used in a training scheme.

20. Use of the apparatus according to claim 1 or the wrist band according claim 16, wherein the electrocardiogram, heart rate and/or heart rate variations are used to determine the occurrence of a heart failure and/or the possibility of occurrence of a heart failure.

21. Method for monitoring a heart of a patient using the apparatus according to claim 1 or the wrist band according claim 16, wherein the electrocardiogram, heart rate and/or heart rate variations are used to determine the occurrence of a heart failure, wherein, upon the determination of a heart failure, the apparatus generates an alarm signal.

22. Method according to claim 21, wherein the alarm signal is sent to an external receiver.

23. Method for monitoring a user's heart, for example using an apparatus according to claim 1, wherein at least one sensor is being positioned at or near the surface of the user's limb, wherein the sensor measures changes in at least one electrical parameter of the user's limb via the surface of the user's limb, wherein detected changes in the at least one electrical parameter of the user's limb are used to determine an electrocardiogram, heart rate and/or heart rate variation of the user's heart, wherein preferably the determined electrocardiogram, heart rate and/or heart rate variation are being stored, sent to a remote location, displayed, and/or made auditively and/or visually knowable to the user, and wherein two capacitor electrodes of a capacitor comprised in at least one of the sensors are placed at positions on substantially opposite sides of the limb, the at least one electrical parameter comprising a permittivity of the limb, and wherein method comprises arranging the capacitor such, that a capacitance of the capacitor is sensitive to pressure pulses in at least one blood vessel of the limb.
